(19) United States
(12) Patent Application Publication
(10) Pub. No.: US 2025/0318951 A1
(43) Pub. Date: Oct. 16, 2025

(54) METHOD OF TREATING AND PREVENTING VIRAL INFECTIONS

(71) Applicants: Vladimir Oks, Medford, MA (US); Andrey Oks, Freehold, NJ (US)

(72) Inventors: Vladimir Oks, Medford, MA (US); Andrey Oks, Freehold, NJ (US)

(21) Appl. No.: 19/250,004

(22) Filed: Jun. 25, 2025

Related U.S. Application Data
(63) Continuation-in-part of application No. 17/403,813, filed on Aug. 16, 2021.
Application No. 17/403,813 is a continuation-in-part of application No. 16/825,441, filed on Mar. 20, 2020, now abandoned.

(51) Int. Cl. A61F 7/00; A61F 7/12

(52) U.S. Cl. CPC A61F 7/0085; A61F 7/12; A61F 2007/0059

(57) ABSTRACT

A method for treating viral infections caused by viruses whose survival time depends on temperature (e.g., cold viruses, flu viruses, COVID-19, etc.) is based on administrating to a subject an effective amount of thermal energy transferred from a hotter liquid product to the colder subject using a predetermined combination of the temperature of the liquid product, the duration of time of supplying the liquid product, and the amount of the liquid product. This method of treatment inactivates viruses in the upper respiratory tract and gastrointestinal tract of the subject and enhances the response of the immune system, thereby preventing complications and accelerating the recovery of the infected subject.

### CROSS REFERENCE TO RELATED APPLICATIONS

This application is a Continuation-in-Part to U.S. patent application Ser. No. 17/403,813, filed Aug. 16, 2021, which is a Continuation-in-Part to U.S. patent application Ser. No. 16/825,441, filed on Mar. 20, 2020, the entire contents of all of which are hereby fully incorporated herein by reference for all purposes.

### FIELD OF THE INVENTION

The present invention relates to the treatment of viral respiratory infectious diseases using a heat therapy method including targeted oral administration of a liquid product with an elevated temperature into the upper respiratory tract and gastrointestinal tract.

### BACKGROUND OF THE INVENTION

A viral infection occurs when a virus enters the body, invades healthy cells, and begins to multiply, often causing illness. Common viral illnesses include colds, flu, COVID-19, norovirus (“stomach flu”), HPV (warts) and herpes simplex virus (cold sores). Many viruses go away on their own, but some cause life-threatening or chronic illnesses. The most common respiratory infection viruses include rhinovirus, influenza virus, coronavirus (e.g. SARS-CoV-2), which cause colds, flu, COVID-19, respectively, with such respiratory infections causing many similar symptoms.

### Common Cold Viruses:

The common cold is a very frequent acute illness in industrialized societies and the leading cause of visits to the physicians in the United States. Though it is usually benign, it is a leading cause of absence from work, also causing a significant economic burden including loss of productivity and treatment costs. The common cold is caused by a variety of viruses, most of the time by rhinoviruses and coronaviruses. However, there is currently no cure for common colds, and the conventional therapy targets the relief of symptoms instead. Prevention strategies for the common cold include avoiding infected people and frequent hand washing during cold season. Conventional therapies have limited efficacy. Certain drugs are costly and have side effects. Dietary supplements (e.g., Echinacea, ginseng, etc.) are believed to be effective by some, but they lack formal studies using modern culture methods.

From U.S. Pat. No. 7,100,605 a device is known, which is constructed in the form of a hand-held hair dryer or a table device for the localized hypothermic treatment of colds. Specifically, “A therapy device for the specifically local treatment of colds by an ionized heated current of air warmed up to at least the human central body temperature and acting directly and in a locally restricted manner via the nose onto the epithelium of the nasal cavities and the adjacent areas in the nasal/throat region . . . ”. The process of inhaling the heated air flow through the nose lasts for 3 minutes “at a temperature of 37° C. to 54° C., preferably of 41° C. to 44° C.” and should be repeated “five times with intervals 1-1.5 hours”. “In this context hot air is inhaled exclusively for approximately 3 minutes, following which approximately 1.5 cups of warm water should be drunk, whereby “warm” means that the water can be swallowed without difficulty in normal mouthfuls and, more significantly, at intervals of 2 to 3 seconds”.

In this context, hot air is inhaled exclusively for approximately 3 minutes because according to the invention, “during long-term application, this can have a negative effect on health and on complete recovery” for various reasons, including electromagnetic fields that hair dryers or a table device resembling a face sauna usually produce, despite the fact that the device housing contains crystallized salt, on which a current of warm air impinges before discharge from the housing resulting in negative ionization of the heated air flow. Any hair dryer that produces heated air limits the value of the treatment by the device using it. According to this invention Opitz teaches “that the personal electromagnetic field of a person suffering from a cold has already become weakened because the body is making energy available for the healing process” and can be disrupted by the electromagnetic fields caused by the hair dryer. Based on general knowledge and a synthesis of scientific literature and consumer technology claims, although use of such a hair dryer with crystallized salt may emit negative ions to reduce positive ions, its electromagnetic effect on respiratory health of people experiencing viral infections remains unsubstantiated.

At the same time, Opitz teaches the consumption of warm water specifically to prevent dehydration, which often accompanies a cold: “This process dehydrates the body by sending an unusually large amount of water into the nasal passages” (as Opitz teaches), especially when inhaling dry, saturated by crystallized salt heated air. Water helps to replenish fluids and helps with dehydration. However, such water consumption taught by Opitz does not kill and/or inactivate viruses, nor is it at all his intention that it does. The 1.5 cups taught by Opitz contain 360 mL of water. “A normal mouthful” of water is generally around 20-30 milliliters (ml) per mouthful for an average adult, while a small sip is 5-10 mL, and a big gulp is 50 mL or more. The upper limit temperature of water that an ordinary person can drink “without difficulty in normal mouthful” is 45-50° C. that feels hot but is generally tolerable, according to scientific sources (Journal of Food Science) and practical observations about human heat tolerance: “50° C. feels very hot, smaller sips needed, but still drinkable carefully”. So, this “normal mouthful” volume does not allow drinking water with a temperature above 50° C. With a volume of 25 mL per gulp (the average amount taught by Opitz), the number of normal mouthfuls is 360/25=14.4 gulps. With intervals of 2-3 seconds and an additional swallowing time of about 2 seconds, the total duration (2.5 sec+2 sec) of the process of drinking warm water is: 14.4×4.5≈65 seconds, or about 1 minute. Opitz also teaches intervals between the first and second intake, between the second and third intake, etc. are 1-1.5 hours or 3600-5400 seconds which represent a break in treatment, during which viruses continue to penetrate cells and multiply. This means that the treatment process taught by Opitz is interrupted and not continuous. Many scientific studies show that the survival time of cold viruses like rhinoviruses and coronaviruses in hot water lose infectivity. For example, at temperatures 60° C. and above, the viruses lose infectivity in under 5 minutes, and at 50-55° C. the viruses may last for up to 10-15 minutes. However, at 40-45° C., the viruses could survive for hours.

Scientific data on coronaviruses (e.g. SARS-CoV-2), shows temperature and/or duration information and the virus's sensitivity to heat are used to provide informed recommendations for thermal inactivation of viruses on masks, clothing, or other objects, stating that the survival time in hot water is: 60° C.—inactivation achieved in 20 minutes; 65° C.—inactivation achieved within 5 minutes; 75° C.—inactivation achieved in 3 minutes. According to scientific publications, 90 minutes (or 5400 seconds) are required to destroy/inactivate viruses in laboratory conditions, and a temperature of at least 56° C. is required for stocks containing viral aggregates. Regarding the spread of viruses inside the human body, a longer thermal effect on the viral infection is most likely required. Therefore, local inhalation devices that additionally use water at a relatively low temperature (below 50° C.) (such as that taught by Opitz), with a short treatment period (about 1 minute), with significant breaks between applications (1-1.5 hours), during which viruses continue to penetrate cells and multiply, cannot transfer enough thermal energy to the upper respiratory and GI tracts to increase the temperature of these areas sufficiently to kill/inactivate viruses. Also, such water consumption does not adequately affect the “inner body” temperature of the patient, nor the «center body” temperature of the patient since they do not boost the heat production of the immune system. The treatment of colds using the said therapeutic device of Opitz is based on the fact that cold viruses, i.e., primarily rhinoviruses and coronaviruses, can be eliminated by actively treating the mucous surface of the nasal and throat passages with heated ionized air flow. However, this process cannot affect the viruses that have penetrated into cells nor prevent their replication. The device of Opitz may provide relief to some cold symptoms such as nasal congestion and sore throat, and thereby make the disease easier to bear, but the treatment does not speed up the recovery process. The temperature and the duration of continuous, uninterrupted treatment are critical parameters for heat therapy for viral inactivation.

There are various applications of heat in the treatment of colds. Certain so-called facial saunas, for isolated treatment of the head area, have been developed for the treatment of colds, and dry saunas have been recommended by so-called holistic therapists. Existing methods with external heat exposure on the body (dry saunas, hot baths, sun, hot air, UV) can help relieve the symptoms of a viral infection, but they do not treat or cure the viral infection itself. The World Health Organization (WHO) and the US Centers for Disease Control and Prevention (CDC) do not recommend these applications as a treatment or preventive measure for viral infections. Moreover, WHO and CDC warn that improper use of such techniques may pose a health risk, may be harmful and have side effects, especially with prolonged use when a person gets sick, and his/her body is weakened and exposed to the stress of a viral infection. Short-term application of external heat does not raise upper respiratory and gastrointestinal GI) tracts temperatures sufficiently, does not affect either “inner body” temperature, and does not affect the core body temperature to inactivate the virus. They may relieve symptoms, “but will not make your cold go away any faster” (CDC Features, Common Colds: Protect Yourself and Others).

### Flu Influenza Viruses

Flu is a contagious illness caused by the influenza virus. Common flu is characterized by an acute infection associated with respiratory problems, intense muscle pain, headaches, chills, nasal obstruction, fever, cough, sneezing and sore throat. The majority of the population will generally recover from flu without any complication. Older people, e.g., young children and people with deficient immune systems, may have serious complications or may even die from the flu.

Current literature describes human common influenza as a serious disease causing an estimated 36,000 deaths each year in the United States alone. Yearly common influenza (flu) epidemics result in lost workdays and schooldays as well as a significant number of hospitalization days and even deaths among the elderly, seniors being particularly vulnerable to respiratory infections.

New influenza strains infecting humans also appear from time to time. These strains frequently originate from other species and have adapted to humans through mutations. The swine influenza A H1N1 flu virus, also named Swine Flu, has been reported around the world and was declared a pandemic influenza virus. Swine flu H1N1 originally only affected pigs but started infecting humans in North America in 2009. Humans have little to no natural immunity to this virus, and it can lead to serious and widespread illness.

Vaccines can prevent flu in 70 to 90% of the cases observed in healthy adults. However, the flu vaccine has to be redesigned each year because the previous year's vaccines are likely to be ineffective against the newly mutated strains. The yearly vaccine is therefore designed before the season begins based on the viruses from the previous year. Since scientists usually cannot predict with accuracy which strain will be dominant, the vaccine may not fully correspond to the new strain. In addition, the flu vaccine is specifically designed against influenza virus strains A and B and does not protect against cold viruses such as rhinovirus and corona types.

Flu symptoms can be alleviated with either the previously mentioned treatment or with, for example, 1) over-the-counter analgesics to relieve pain and reduce fever; 2) cough suppressants for dry cough with no mucus; 3) expectorants to help clear mucus so it can be coughed up; and/or 4) decongestants to reduce nasal congestion.

### Severe Acute Respiratory Syndrome Coronaviruses

Coronaviruses vary significantly in risk factors. Some can kill more than 30% of those infected and some are relatively harmless, such as the common cold. Coronaviruses cause colds with major symptoms, such as fever, and sore throat from swollen adenoids, occurring primarily in the winter and early spring seasons. Severe acute respiratory syndrome coronaviruses like SARS-CoV, COVID-19 can cause pneumonia (either direct viral pneumonia or a secondary bacterial pneumonia) and bronchitis (either direct viral bronchitis or secondary bacterial bronchitis) in any season during the year and in most any climate. The much-publicized human coronavirus discovered in 2003, SARS-CoV, which causes severe acute respiratory syndrome (SARS), has a unique pathogenesis because it causes both upper and lower respiratory tract infections.

Scientific publications provide data on the dependence of virus survival time on temperature and humidity. For example, Coronavirus Survival Times at Relative Humidity & Air Temperatures in Hours & Days (K. H. Chan, 2011; Smith RD, 2006) describe the following:

The survival times at 80% RH and 40° C. were less than 7 hours for proxies of coronaviruses on stainless steel.

The survival times at 50% RH and 40° C. were more than 24 hours. There was a reduction in the ratio of virus to −3 Log 10 (Nt/N0) in 24 hours.

The survival times at 20% RH and 40° C. were more than 120 hours. There was a reduction in the ratio of virus to −3 Log 10 (Nt/N0) in 120 hours.

The survival times at 50% RH and 20° C. were less than 7 days on stainless steel.

Some scientific publications showed that heat treatment of SARS-CoV for 45 min at 75° C. resulted in inactivation of the virus, while 90 min at 56 and 65° C. was required for virus inactivation (Darnell, M. E. R., et al., Inactivation of the coronavirus that induces severe acute respiratory syndrome, SARS-CoV, 2004). This publication states that “data are similar to the findings of Duan et al. (2003), wherein viral inactivation occurred at 90, 60, and 30 min after incubation at 56, 65, and 75° C., respectively. Heat is an effective means of SARS-CoV inactivation; however, stocks containing viral aggregates may require a longer duration of heat exposure”.

Abraham's publication shows independent studies, the results of which can be used to formulate general guidelines.

Table 1 below shows viruses that may be treated using the present method, with the table also showing exposure temperatures and associated durations of time for inactivating coronavirus strains:

TABLE 1

| Temperature | Duration | Log | |
|---|---|---|---|
| (° C., ° F.) | (minutes) | reduction | Virus |
| 56, 133 | 10 | 5 | SARS-CoV (Urbani strain) |
| 56, 133 | 20 | 6 | SARS-CoV (Urbani strain) |
| 65, 149 | 3 | 6 | SARS-CoV (Urbani strain) |
| 75, 167 | 15 | 7 | SARS-CoV (Urbani strain) |
| 55, 131 | 120 | 5 | Gastroenteritis coronavirus |
| 56, 133 | 60 | 7 | Canine coronavirus |
| 65, 149 | 40 | 7 | Canine coronavirus |
| 75, 167 | 15 | 7 | Canine coronavirus |
| 56, 133 | 50 | 5 | Canine coronavirus |
| 65, 149 | 5 | 5 | Canine coronavirus |
| 75, 167 | 4 | 5 | Canine coronavirus |
| 56, 133 | 30 | >5 | SARS CoV, FFM1 no protein |
| 50, 122 | 30 | 1.9 | SARS, CoV, FFM1 With 20% |
| | | | protein |
| 60, 140 | 30 | >5 | SARS CoV, FFM1 no protein |
| 60, 140 | 30 | >5 | SARS, CoV, FFM1 With 20% |
| | | | protein |
| 56, 133 | 5 | 5.8 | SARS CoV (Hanoi strain) |
| 56, 133 | 10 | 6.5 | SARS CoV (Hanoi strain) |
| 56, 133 | 30 | >6.4 | SARS CoV (Hanoi strain) |
| 56, 133 | 30 | 2-5 | SARS CoV (FFM1 strain) |
| 56, 133 | 20 | >4.3 | SARS CoV (Urbani strain) |
| 60, 140 | 30 | >5 | SARS CoV (FFM1 strain) |
| 60, 140 | 30 | >4 | SARS CoV (FFM1 strain) |
| 60, 140 | 60 | >4 | SARS CoV (FFM1 strain) |
| 65, 149 | 10 | >4.3 | SARS CoV (Urbani strain) |

Although no studies have been published on the resistance of the Covid-19 coronavirus to high temperatures, it is likely that they will be similar to the results in the table, which can be used to formulate general guidelines. Different scientific papers provide for different variants of viruses different data, but the trend of the survival time of the coronavirus depending on temperature and humidity remains.

Viruses spread from person to person primarily via respiratory droplets from coughing or sneezing. People are thought to be most contagious when symptomatic, though some spread might be possible before symptoms show. The time between exposure and symptom onset is typically five days but may range from two to fourteen days. Common symptoms include fever, dry cough, and shortness of breath. Complications may include pneumonia and acute respiratory distress syndrome. There is no specific antiviral treatment, except vaccines that are likely to be ineffective against the newly mutated strains, but research is ongoing. Efforts are aimed at managing symptoms and supportive therapy. Recommended preventive measures include handwashing, wearing facemask, using hand sanitizer, maintaining distance from other people (particularly those who are unwell), and monitoring and self-isolation for fourteen days for people who suspect they are infected.

Public health responses have included national pandemic preparedness and response plans, travel restrictions, quarantines, curfews, event postponements and cancellations, and facility closures. These include a quarantine of Hubei, China; the nationwide quarantines of Italy, Spain, the Czech Republic, and Germany; curfew measures in China and South Korea; various border closures or incoming passenger restrictions screening methods at airports and train stations; and travel advisories regarding regions with community spread.

Symptoms of COVID-19 are non-specific and those infected may either be asymptomatic or develop flu symptoms such as fever, cough, fatigue, shortness of breath, sore throat, or muscle pain. Further development can lead to severe pneumonia, acute respiratory distress syndrome, sepsis, septic shock and death. Some of those infected may be asymptomatic, returning test results that confirm infection but show no clinical symptoms, so researchers have issued advice that those with close contact to confirmed infected people should be closely monitored and examined to rule out infection.

There is a need for a method that can be useful for the treatment and prevention of viral infections caused by viruses of cold, flu viruses and coronaviruses of SARS-CoV 1, 2, which is safe, easy to use and has advantages over existing methods of treatments.

The inventive method described herein is based on the theory of anthropology science, studying the process of historical and evolutionary formation of humans. The method is a natural extension of human evolutionary development, since the human use of fire, in the fight for its existence, in the fight against foreign bodies in an organism including in the fight against viruses.

This method is subject to the science of biology, the natural science that studies life and living organisms, especially its immunology branch that covers the study of immune systems in all organisms by the physiological functioning of the immune system in both state of health and state of illness, and the body's response to foreign bodies. The main purpose of the immune system is to protect against intrusions and to preserve the integrity of the internal environment of the body, its biological individuality.

This method is subject to the laws of cybernetics, the science of general patterns of information management and transmission processes in machines, living organisms and society. Cybernetics includes the study and impact of feedback on management and communication in living organisms, machines, and organizations, including self-organization, and regulation of all processes occurring in nature.

This method is subject to the laws of mechanics—a section of physics, a science that studies the movement of material bodies and the interaction between them. Man is the most complex object not only on Earth, but perhaps in the entire Universe. Given the evolutionary development of man and the progress of modern technology, man can be considered as a material, mechanical object. The functioning of individual parts and systems of the human body from the point of view of physics, physiology, biology and microbiology can be presented in a simplified form and described by a small number of differential equations.

This method follows the laws of energy, a branch of physics that studies the quantitative property of energy that must be transferred to an object to perform work or heat the object. Energy is a conserved quantity; the law of conservation of energy states that energy can be transformed into a form but not created or destroyed. This method follows the laws of transfer of chemical energy released when a fuel burns, as well as the thermodynamic transfer of thermal energy due to the temperature of the object.

This method is subject to the science of chemistry—the discipline, which is involved with the structure, properties and behavior of elements, compounds and the changes they undergo during a reaction with other substances, specifically its sub discipline of biochemistry's field of metabolism, the set of life sustaining chemical reactions in organisms.

This method is subject to the law of continuity of processes in time, the main property of which is the existence of a certain value of the function describing these processes at any moment in time. The functioning of many processes in nature, including in living organisms, is carried out in an integral dependence on changes under the influence of internal and external impacts.

### SUMMARY OF THE INVENTION

As is known, a human is a very complex living subject that reacts to external and internal influences with the help of feedback signals received by the sensitive elements of the human body. The speed of processing the received information and the speed of the human body's response to these effects is very diverse, but mostly relatively slow. For example, the transformation of chemical reactions occurring in the human body into thermal energy that ensures human life activity occurs continuously, quite slowly and is in integral dependence on external and internal changes. Such a function of the human body as maintaining and regulating body temperature is carried out by numerous processes occurring in the body such as metabolism, endocrine and hormonal regulation, etc., and in the event of a violation of the body's vital activity by a viral infection, the immune system actively counteracts foreign pathogenic bodies. The purpose of the present invention is to help the body as a whole and the immune system in particular in the fight against a viral infection.

This method of treating a viral infection caused by viruses in a subject in need thereof comprising administering to the subject an effective amount of transferred thermal energy from a hotter liquid product to a colder subject comprising a predetermined combination of the temperature of the hot liquid product, duration of heat transfer of the liquid product, and sufficient amount of liquid product consumed orally to produce over a time warm-up effect of the subject upper respiratory tract and gastrointestinal (GI) tract temperature, to boost the immune system reaction, and whereby the infection is treated.

With the present method, the liquid product (agent) delivering heat is hot, pre-boiled, drinking water—the simplest, cheapest and most natural product for a human. The thermal energy supplied for heat exchange is produced by consuming hot water in the upper respiratory and GI tracts. Water is the most acceptable liquid product for the human body that can create such conditions for the environment in the upper respiratory and GI tracts. The consumption of hot water for a long period of time with high temperature inactivates/kills viral infection. Hot water delivers much more oxygen to the body, improves blood circulation, improves metabolism, reduces painful contractions of muscles, relieves nasal congestion, aids digestion, calms central nervous system, helps relief constipation, helps reduce toxins, and keeps you hydrated.

Water is the most common fluid used for heat exchange, due to its availability and high heat capacity. It is especially effective to transport heat that will flow from the hot environment to the cold one in an attempt to equalize the temperature difference. This method works similarly to the heat transfer mechanism in a heat exchanger, where one can determine the thermodynamic entropy for an isolated system, which serves as a measure of how energy is spread out or dispersed in a system and reflects the quality of the energy—how useful it is for doing work. During some period of time of consuming hot water, the temperature of the upper respiratory and gastrointestinal tracts becomes higher than the core body temperature, since the energy of hot water is dissipated from the injection point throughout the body, and as the entropy of the water decreases, some of its energy is transferred to the human body, whose entropy increases.

The goals of the present method of treating and preventing viral infections are to:
1. Inactivate/kill viruses in the upper respiratory and GI tracts,
2. Prevent complications, especially pneumonia,
3. Expedite recovery, and
4. Prevent viral diseases.

The method of treating and preventing viral infections of the upper respiratory tract is based on a fact that virus's survival time depends on the temperature and an assumption based on scientific research that a virus can be inactivated/killed or its ability to penetrate and replicate copies inside living cells can be weakened, by heat exposure to the virus. Creating an environment with elevated temperature in the upper respiratory and GI tracts and preventing spread of the virus into the lower part of the respiratory tract using long-term, continuous, without breaks exposure of heat, prevents development of pneumonia and accelerate recovery.

It is known that the survival time of the virus depends on the temperature and that the temperature affects the viability of different types of viruses in the same way—the higher the temperature, the shorter the survival time of viruses. The higher the temperature and the longer the heat exposure to the virus, the greater the possibility of inactivation of the virus. The present invention is designed to create an elevated temperature environment in the upper respiratory and GI tracts, where respiratory viral infection is mainly concentrated.

The present method of treatment fights different unique viruses and their variants and contains the ability for each user to individually select the duration, temperature, speed, and volume of hot water consumption that corresponds to the unique characteristics of viruses, creating a unique protocol, requiring unique combination of variables.

Inactivation of various viruses is achieved by the available range of hot water consumption durations between the minimum duration and maximum duration of hot water consumption and the available range of hot water consumption volumes between the minimum volume and maximum volume of hot water consumption. The parameters of heat energy transfer are calculated depending on the temperature of the liquid product consumed and the weight of the patient requiring treatment using the inventive formulas proposed by this method. These ranges allow the correct selection of variable parameters for the treatment of mild and severe illnesses caused by cold, flu and severe acute respiratory syndrome (SARS) coronaviruses. Each user personally chooses a specific duration, a specific volume, a specific speed of hot water consumption at a specific temperature for a specific viral infection based on his/her needs. A single user can create many different combinations of treatment durations to inactivate different viruses whose survival time is dependent on temperature depending on their individual circumstances.

This method further reduces the risk of pneumonia by washing viruses away from the respiratory tract to the GI tract. At the same time, hot water prevents development of a viral infection in the GI tract. The present method prevents and/or reduces the spread of a virus into the lower respiratory tract and prevents development of pneumonia most effectively at the early stages of an illness when an infection just starts to develop in the upper respiratory and GI tracts. A virus can be killed or its ability to penetrate and replicate copies inside living cells can be weakened, using continuous and prolonged hot water exposure on the virus.

The present method works similar to existing methods of application for medications. Traditional medications typically utilize a particular chemical compound or composition to kill a virus, or boost resistance to a virus, or create an immune system antibody, or aim to relieve symptoms of a disease. There are common rules for the use of any medication. For example, once a person has caught a virus, their personal, unique reaction to the virus may include a fever of 38.9° C. To manage the fever, a doctor may recommend taking a fever-reducing medication. The goal is to manage and return the body temperature to normal human temperature. What medicine, how many tablets, in what dose, for how long, how often the drugs are to be taken, etc., to bring the temperature to normal is recommended by doctor individually to each patient, in accordance with his constitution, one of the main parameters of which is the patient's body weight.

It would seem that if the medications work so well, then it would make sense for a patient to simply take the medications continuously for a long time until he/she is completely cured. However, there are limitations, primarily due to the fact that drugs are chemical compounds that have side effects and that in large quantities may harm and/or even kill a person.

The present method is based on a completely different approach, with the following specified parameters:
1. Apply this method as soon as symptoms begin,
2. Drink hot water with the personal maximum temperature that can be drank using small sips,
3. Drink continuously, with selected consumption rate, intervals between sips, and without stops or breaks,
4. Drink over a long period of time with selected at least minimum duration, with specified maximum duration restrictions, and
5. Beyond the stated restrictions, drink until symptoms disappear or decrease and the patient feels better based on the objective measures and subjective indicators.

This method involves the use of hot water as a liquid medicine for the patient, long-term and continuous consumption of which does not have side effects; therefore, it allows for long-term continuous treatment until complete recovery, or at least until a significant improvement in health. Therefore, the end of the treatment process is determined by:
1. Objective measures, including a decrease in body temperature and stabilization below 37.3° C., a reduction in sore throat below 2 points on a scale of 0 to 10, and/or a reduction in muscle aches below 2 points on a scale of 0 to 10, or a reduction headache below 2 points on a scale of 0 to 10, normalization of individual's pulse rate; and
2. Subjective indicators that indicate to the patient a complete disappearance of symptoms or a significant reduction in symptoms, including no longer experiencing chills, relief of nasal or chest congestion, relief of cough, ease of removing infected mucus, more comfortable breathing, feeling less tired and more energetic, and improvement in general well-being of at least 70%.

The proposed method of treatment is carried out in accordance with the inventive formulas described herein for determining the main parameters of hot water consumption, also depending on the constitution of each person.

The present method utilizes temperature and time to kill or weaken most respiratory virus. If any respiratory virus is exposed to boiling water, it will be killed within seconds with inactivation times varying for cold viruses, flu viruses, coronaviruses. Each viral infection corresponds to a unique virus that responds to a unique therapy for each person or group of patients.

Each patient should drink hot water using the present method according to the person's unique physical constitution (for example weight), biological, genetic characteristics, health condition, which includes processes of works of metabolism, immune system, kidney, liver, heart, as well as the type of virus, strain, stage of viral infection, symptoms, season, climate, uniqueness of the population. For example, in China, the average weight of the population is less than the average weight of the population in the United States.

This method uses regular drinking water but if necessary, and as recommended by a physician, natural organic chemical compounds derived from herbs, fruits, minerals, and other sources can be added to water in small amounts to help maintain pH balance and combat viral infections. These compounds not only help maintain a healthy pH, but also have antiviral, immune-boosting, and anti-inflammatory properties.

As mentioned above, this method uses pre-boiled drinking water. It is preferable to use boiled water, since when drinking large volumes of water, it should be free of or with a minimum number of viruses, bacteria, and parasites, especially since the patient's body is weakened. Water may be boiled in regular teapots, coffee machines, heating elements or over an open fire.

It is known that hot coffee and tea drinks are brewed at temperatures high enough to cause immediate and serious burns. On the other hand, numerous studies have been conducted to determine the preferred temperatures for consuming hot drinks. The result of these complementary studies is that the preferred drinking temperatures are significantly lower than the brewing temperatures. Therefore, there is a great need to differentiate between the brewing temperatures and the drinking temperatures of hot drinks.

A rationally recommended range of serving temperatures in the US is 131° F. (55° C.) to 165° F. (71° C.). This recommendation balances the range of consumer preferences and safety. The temperatures listed are based on general industry standards, food safety recommendations, and research from sources such as tea and coffee industry recommendations (e.g., National Coffee Association, Tea Association of the USA), food safety guidelines (e.g., FDA, USDA), scientific studies on optimal drinking temperatures, barista and tea expert recommendations, etc. The range used for serving hot drinks worldwide is between 55° C. and 75° C.

Thus, the water temperature of 131° F. (55° C.), chosen as the minimum temperature for this method, is safe for human drinking from a cup/mug. The best cup/mug to use for this method is a double-walled insulated cup/mug, which prevents heat from transferring to the outer walls, keeping the drinks hot.

A well-insulated double-walled mug can keep water hot (near its original temperature) for about 4-6 hours, and warm for up to 12 hours. Premium brands like Yeti®, Hydro Flask®, or Thermos® often perform at the higher end of this range. Table 2 below shows manufacturer's specifications for the specific mugs “Heat Retention Estimates (55° C. to 75° C. Range)”, with and without a lid.

TABLE 2

| Mug Type | With Lid | Without Lid |
|---|---|---|
| Vacuum-Insulated Stainless Steel | 4 to 6 hours | 2 to 4 hours |
| (e.g., Thermos ®, Yeti ®, | | |
| Zojirushi ®) | | |
| Double-Wall Glass (Non-Vacuum) | 1 to 2 hours | 30 minutes to 1 hour |
| Double-Wall Ceramic (Non-Vacuum) | 1 to 2 hours | 30 minutes to 1 hour |
| Double-Wall Plastic (Non-Vacuum) | 1 to 3 hours | 30 minutes to 1 hour |

The efficiency of using hot water as a heat exchange agent for heat exposure on the virus is much higher than the efficiency of supplying warm air flow used for inhalation. This method determines aspects of the influence and interaction of a patient and the virus, taking into account the patient's physical, physiological, biological, microbiological, and chemical characteristics, as well as the influence of the environment, the temperature of hot water and the exposure time.

Furthermore, viral infections occurring in the rectum and whose survival time depends on temperature also can be treated using this method by means of a prolonged continuous-discrete supply of a heated liquid product (e.g., by pure water). Devices necessary for carrying out such procedures may be specially developed.

In addition, viral infections on the human body (HPV) and facial herpes simplex (cold sores on or around the lips) also can be treated in a similar manner. For this purpose, a sterile soft material (e.g., gauze pads, cotton balls) soaked in a hot liquid, at least clean water with a temperature higher than the normal human body temperature, which is the maximum permissible temperature that does not cause a burn, can be used directly to the infected area for an extended period of time, continuously-discretely.

According to one aspect, one or more embodiments are provided below for a system and method for treating viral infections. The method may include a method of treating a viral infection in a human subject in need thereof, the viral infection caused at least by a contagious respiratory coronavirus related to severe acute respiratory syndrome (SARS) coronavirus whose survival time is temperature dependent. The method includes administering to the subject an amount of thermal energy from a liquid product using a predetermined combination of (i) a temperature of the liquid product, (ii) a duration of time of the administrating the thermal energy from the liquid product, and (iii) an amount of the liquid product, wherein the temperature of the liquid product is at least 55° C., and the liquid product is at least pre-boiled drinking water.

In another embodiment, the thermal energy is administered orally to an upper respiratory tract and a gastrointestinal tract of the subject by having the subject drink the amount of the liquid product.

In another embodiment, the subject drinks the amount of the liquid product at a consumption rate, wherein the consumption rate is based on a body weight of the subject.

In another embodiment, the consumption rate is determined by adding a first constant value to a product of the body weight of the subject and a second constant value, wherein the first constant value is 14 mL/min, and the second constant value is 0.34 mL/min/kg.

In another embodiment, the subject drinks the amount of the liquid product in sips, each sip having a sip volume, wherein the sip volume depends on the body weight of the subject and is determined by adding a third constant value to a product of the body weight of the subject and a fourth constant value, wherein the third constant value is 2 mL, and the fourth constant value is 0.06 mL/kg.

In another embodiment, the subject drinks the sips sequentially with an interval between each sequential sip, wherein the interval is determined by subtracting a fifth constant value from a quotient of the sip volume and the consumption rate, wherein the fifth constant value is 2 seconds.

In another embodiment, the temperature of the liquid product is determined as a maximum temperature of the liquid product, wherein the maximum temperature of the liquid product is a highest temperature of the liquid product that the subject drinks in sips having the predetermined sip volume without causing scalding of the subject, wherein the maximum temperature of the liquid product is greater than or equal to a sixth constant value, wherein the sixth constant value is 55° C.

In another embodiment, the subject drinks the amount of the liquid product for at least a minimum duration of time based on the maximum temperature of the liquid product.

In another embodiment, the minimum duration time is determined by dividing a seventh constant value by the maximum temperature of the liquid product, wherein the seventh constant value is 5850 min x° C.

In another embodiment, the subject drinks the amount of the liquid product for no longer than a maximum duration of time determined by adding an eighth constant value to the minimum duration of time, wherein the eighth constant value is 45 min.

In another embodiment, the amount of thermal energy administered to the subject is based on the product of the maximum temperature of the liquid product and the amount of the liquid product, wherein the amount of the liquid product is a volume of the liquid product.

In another embodiment, the subject drinks the liquid product for at least the minimum duration of time, during which the subject drinks at least a minimum volume of the liquid product, wherein the minimum volume of the liquid product is determined by multiplying the minimum duration of time by the consumption rate.

In another embodiment, the subject drinks no more than the maximum volume of the liquid product determined by multiplying the maximum duration of time by the consumption rate.

The presently disclosed system and method for evaluating growing media is more fully described in the detailed description below.

### Objectives of the Present Method

An objective of the present invention is to create an environment with an elevated temperature in the upper respiratory tract and gastrointestinal tract to prevent the spread of the virus to the lower respiratory tract and prevent the development of pneumonia.

It is an objective of the present invention to expedite recovery.

An objective of the present invention is to use this method universally as an applicable treatment against the above-mentioned viruses, the survival time of which depends on temperature.

It is an objective of the present invention to administer an affordable liquid product and all the means necessary to use this treatment method at home to millions of people without drugs or special equipment.

Another objective of the present invention is to use this method as a preventive measure, e.g., similar to the recommendation to “wash your hands”.

A further objective of the present invention is to provide a safe treatment method with little to no side effects.

An additional objective of the present invention is to use at least hot drinking pre-boiled water as a liquid product heat carrier—the simplest, most inexpensive, and most natural product for humans.

A further objective of the present invention is to provide all the preparations that allow the method to be used anywhere, so that the user can continue the therapy for a day, days, weeks and/or months.

It is another objective of the present invention to reduce the number of frequent acute illnesses causing a significant economic burden including loss of productivity, treatment costs, and to help slow down the spread of the cold, flu, coronaviruses epidemics or pandemics.

It is also an objective of the present invention to treat and prevent norovirus infection, as well as to help inactivate/kill other foreign pathogenic bodies within the GI tract.

Another objective of the present invention is to open a new chapter in the study of the use of hyperthermia using liquid products for the treatment of diseases caused by other foreign pathogenic bodies in the human body, including some types of herpes simplex, HPV, or cancer cells, as well as the creation of special medical devices for carrying out therapeutic and prophylactic procedures.

### BRIEF DESCRIPTION OF THE DRAWINGS

The study of this method is based on one of the most important parameters of human health—human core (body) temperature and regulation of fever, the most common symptom of viral infectious diseases. The thermoregulation model presented below is a feedback control system under the influence of viral infection and the effect of this method.

In general, there are many different types of feedback control systems including those based on step response time: fast response—microseconds to seconds; intermediate response—seconds to minutes; long-term adaptation—minutes to hours or even days. Most feedback control systems use traditional proportional-integral (PI) controllers.

The advantages of the present invention will become apparent from the following description of the accompanying drawings, in which:

FIG. 1 illustrates a simplified model of a human body temperature regulation system that is similar to a block diagram of feedback control system with a conventional proportional and integral (PI) controller.

FIG. 2 illustrates a simplified model of a human body temperature regulation system under influence of a viral infection as a foreign pathogen, introduced into a human organism that is similar to a block diagram of feedback control system with a conventional PI controller and external source of disturbance.

FIG. 3 illustrates a simplified model of a human body temperature regulation system under the influence of a viral infection as a foreign pathogen into a human organism and usage of the present invention that is similar to a block diagram of feedback control system with a conventional PI controller, external source of disturbance and disturbance compensator.

FIG. 4 shows steps that may be taken to determine a personal maximum water temperature (PMWT) according to exemplary embodiments hereof.

FIG. 5 shows steps that may be taken to perform a viral infection treatment method according to exemplary embodiments hereof.

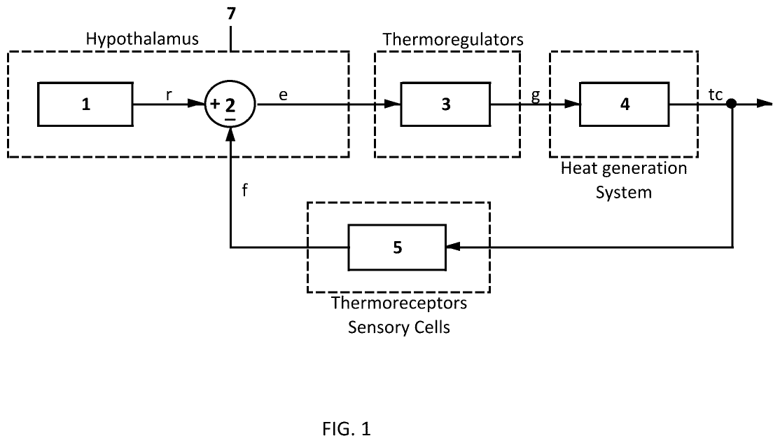

### DESCRIPTION OF THE FIG. 1

FIG. 1 shows a block diagram of feedback control system.

In some embodiments, as shown in FIG. 1, a set point unit 1 generates the input setting signal r, which is compared with the negative feedback signal f by the block of summing 2, which in this case calculates the difference between the signals r and f to obtain the deviation signal e. The deviation signal e is input to the PI controller 3. Controller 3 accordingly generates a variable signal g. The signal g is fed to the input of the actuator block 4. Block 4 generates the output signal tc, which is a controlled output function corresponding to the set signal r. The output signal tc is input to the feedback sensor, block 5. The output signal of the feedback f is fed to the negative input of summation block 2. This is the canonical form of the block diagram of a feedback control system with a conventional PI controller.

The output function of the PI controller is represented as:

$$g ( t ) = ( Kp * e ( t ) + Ki * \int e ( t ) * dt ) \qquad ( 1 )$$

$$where :$$

$$t is a time ;$$

$$Kp is a gain of the P part controller 3 ;$$

$$and$$

$$Ki is a gain of the I part controller 3$$

To simplify calculations, the output function of a feedback control system describing the system's response in terms of its input may use the Laplace transform. In a feedback control system, the transfer function represents the mathematical relationship between the system's input and output in the Laplace domain. The transfer function of the PI controller is represented as:

$$G ( s ) = ( Kp + Ki / s ) * E ( s ) \qquad ( 2 )$$

$$where :$$

$$s is Laplace operator ;$$

$$G ( s ) is the Laplace transform of the output of the controller 3 ;$$

$$E ( s ) is the Laplace transform of the input of the controller 3 ;$$

$$Kp is a gain of the P part ;$$

$$Ki is a gain of the I part wherein \text{:} Ki = 1 / Ti , where Ti is a time constant of integration of integral controller 3.$$

### Model of a Human Body Temperature Regulation

FIG. 1 also illustrates a simplified model of a human body temperature control system. Human core temperature thermoregulation is a complex system that comprises many controllers (regulators), actuators with multiple feedback loops. A complex block diagram of a feedback control system can be simplified using the block reduction rules (e.g., Mason's gain formula for signal flow graphs) to a canonical form with equivalent transfer functions. For high-order systems, representing them as state-space equations instead of block diagrams simplifies analysis. The human body uses two main types of signals to communicate internally: electrical and chemical. These signals work together to control everything including body temperature.

The human brain is the center of the nervous system. The brain exerts centralized control over a body's nervous system and other organs by generating patterns for body activity. This centralized control allows rapid and coordinated responses to changes of internal and external conditions. Complex, purposeful control of the organs and their functioning is based on the brain receiving sensitive information. The center of regulation of human body temperature is the hypothalamus, located in the brain. This is the area responsible for regulating many vital functions, including body temperature.

Hypothalamus 7 of the human brain includes set-point area 1 and the area for processing information received from set-point and feedback signals, which compares them as a comparator 2. In this case, it is the center of the thermoregulation of the human body. The core temperature of a human is regulated primarily by Hypothalamus 7. When the core temperature varies from the homeostatic set point, the endocrine system and autonomic nerve system initiate physiological control mechanisms as part of homeostasis process as needed to return the temperature toward the set point.

Human body temperature can be measured in a few different ways, depending on the method and the tool used: rectally (in the rectum), orally (in the mouth under the tongue), axillary (under the armpit), temporo-arterially (scanning of the forehead surface), or tympanic (ear) thermometers. The core temperature, which in the present invention is defined as the body temperature, most accurately is determined through rectal measurements. The rectal temperature is expected to be approximately 0.55 Celsius (or 1 Fahrenheit) degree higher than an oral temperature taken on the same person, at the same time, at the normal conditions. When using this method of treatment, oral temperature measurement should be performed after some time has passed since its application, so that the temperature in the mouth cools down and becomes equal to the body temperature. Other tools can be used at any time of application of this method.

Thermoreceptor Sensory Cells 5 are free nerve endings or neurons, which are located mainly in the skin, internal organs, skeletal muscles, brain, spinal cord, transmit sensory information f to comparator 2 of Hypothalamus 7. This feedback information signal f is fed to the input of comparator 2 of Hypothalamus 7 with the opposite sign to the setpoint 1 signal r and is negative feedback, stabilizing the normal body temperature tc of a healthy person.

The thermoregulation system of the human body in response to any internal or external changes activates either heat-warm or heat-loss mechanisms, mainly through deep organs, especially through the work of the liver, heart, blood circulation, through metabolism, functions of the endocrine system, immune system, hormonal regulation, through the mechanisms of shivering, sweating, vasoconstriction, vasodilation, all of which is Heat Generation System 4.

As mentioned above, human body temperature thermoregulation is a very complex system that includes many regulators and actuators with multiple feedback signals. The reaction of cells, organs and mechanisms that regulate human body temperature changes varies in power and duration—the response time of different mechanisms of Heat Generation System 4. For example, vasoconstriction reduces heat loss by narrowing blood vessels and lasts for several minutes after a viral infection is detected. Once the fever reaches its peak, vasoconstriction stabilizes. When the infection is controlled and the fever subsides, vasodilation is triggered, which widens the blood vessels and initiates sweating, which releases excess heat. Shivering, as part of the body's response to fever, usually begins within minutes of detecting a viral infection and stops once the fever reaches a new point and the body stops shivering. If the fever subsides, this leads to sweating and vasodilation to cool down. The speed of hormonal action depends on the type of hormone and its target function. Some hormones act within seconds, while others take minutes, hours, or even days to produce effects. For example, the endocrine system releases thyroid hormones T3 and T4 that act during hours and days to increase metabolism and heat production. Metabolism plays a critical role in fighting viral infections by supporting immune function, energy production, and cellular defense mechanisms. When the body detects a viral infection, metabolic pathways adjust to provide the necessary energy and resources for the immune system to fight the infection. Metabolism begins working immediately when the body detects a viral infection, but the speed and intensity of metabolic activation depend on the body's immune response and energy demands. For some metabolic functions this takes minutes, for some it takes hours, and for some it takes days.

Therefore, in this model of human body temperature regulation, the transfer functions of the action time are allocated into Thermoregulators 3. Thermoregulators 3 are combined controllers that control the mechanisms of Heat Generation System 4. The body's temperature regulation involves fast, moderate, and slow mechanisms working together to maintain homeostasis or responding to the viral infection. The transmission time of the regulation signal from Hypothalamus 7 to Thermoregulators 3 is carried out by fast response while Thermoregulators 3 transfers most of the information by moderate and/or slow response with transfer functions for each regulator similar to integral (I) controller of a conventional feedback control system.

The output integral information signal g of Thermoregulators 3 is regulated gradually and continuously. The rate of change of the output signal g is determined by a parameter called the time constant of integration for each regulator and for each person this is their own physiological internal constants. This output signal g enters Heat Generation System 4, which maintains or changes body temperature tc.

For example, setpoint 1 of Hypothalamus 7 gives a command to set the body temperature at 36.7° C. The feedback of Thermoreceptors Sensory Cells 5, measuring the body temperature tc generates a negative feedback information signal f, which is compared with the setpoint signal r by the comparator 2 of Hypothalamus 7. For a healthy person, the feedback signal f is equal to the set signal r, therefore the deviation information signal e of the comparator 2 is zero and there is no change of the information signal g. Heat Generation System 4 maintains the body temperature tc, which corresponds to the command signal r of the setpoint 1 of Hypothalamus 7—36.7° C.

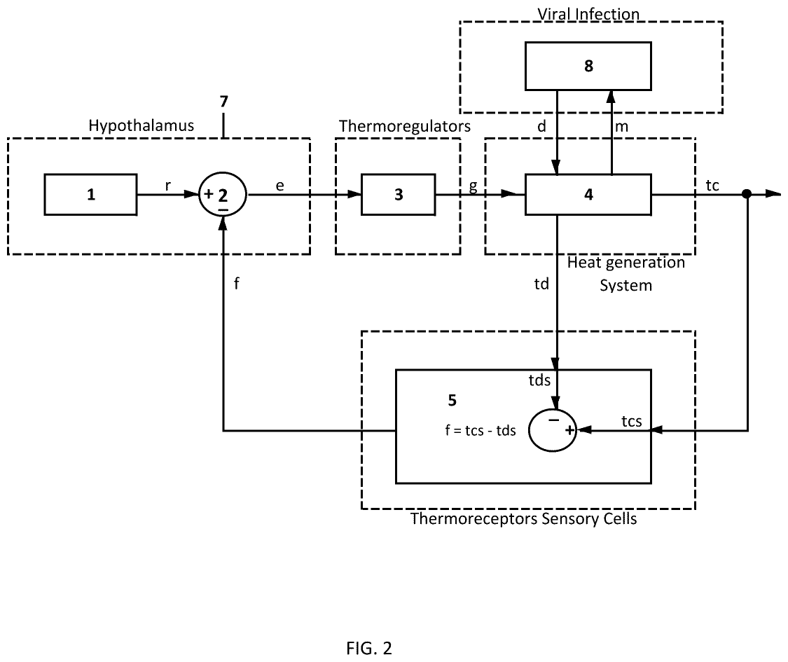

### DESCRIPTION OF THE FIG. 2

FIG. 2 shows the feedback control system of FIG. 1 with an external source of disturbance added, in this case, the presence of foreign pathogen viruses in the human body. As shown, FIG. 2 includes the block diagram of a feedback control system with a conventional I controller and the external source of disturbance block 8 which generates a disturbance signal d that is input into the controlled block 4.

### Model of a Human Body Temperature Regulation in Viral Disorder

FIG. 2 shows the model of the human body's thermoregulation system when exposed to a viral infection as a foreign pathogen, where the source of disturbance is Viral Infection 8, which directly affects Heat Generation System 4 with action d. At the same time, Heat Generation System 4 directly reacts to the disturbance source, Viral Infection 8, especially with the immune system reaction m, as the human body's response to the viral infection.

Heat Generation System 4 under the influence of the action d of Viral Infection 8 releases hormones td, which are transformed by Thermoreceptor Sensory Cells into sensitive information tds with the opposite effect to the sensitive information tcs received under the influence of changes in body temperature tc. The information signal tds have the opposite effect to the body temperature information signal tcs since it represents the viral infection action d, which is disturbing, destabilizing action of Viral Infection 8. Thermoreceptor Sensory Cells 5 process two information signals: the body temperature information signal tc and the viral disturbance temperature information signal td. The information signal td disrupts the normal operation of Thermoreceptor Sensory Cells 5 by signal tds reducing the stabilizing effect of the informational signal tcs of feedback signal f.

At the onset, Viral Infection 8 disturbs the body's homeostatic temperature balance with action d, disrupting the vital activity of cells, the work of certain organs and the functioning of the mechanisms of Heat Generation System 4. On the other hand, Heat Generation System 4 counteracts viruses primarily by the thermal and chemical reactions of the immune and endocrine systems to Viral Infection 8 by reaction m. This is a natural resistance to changes in the normal functioning of internal, physiological, and chemical processes maintained by living organisms. Heat Generation System 4, under the influence of viral infection action d, creates the temperature disturbance information signal td, which reduces the activity of Thermoreceptor Sensory Cells 5 transforming their activity into sensitive information signal tds, thus suppressing the activity of the sensitive information tcs obtained from the signal tc of the human body temperature. The resulting signal of Thermoreceptor Sensory Cells 5 is the feedback signal f.

Under the influence of the disturbance source of Viral Infection 8, the feedback signal f of Thermoreceptor Sensory Cells 5 decreases. The deviation signal e of the comparator 2 of Hypothalamus 7 increases, since the setpoint signal r becomes greater than the negative feedback signal f. The output integral information signal g of Thermoregulators 3 changes gradually and continuously. The rate of change of the output signal g, determined by the integration time constants, depends only on the difference between the signals r and f in accordance with an integral property. This signal g is fed to Heat Generation System 4, which activates heat-warm mechanisms in accordance with the signal g of Thermoregulators 3, and gradually and continuously increases the human body temperature tc.

The body temperature tc increases until the feedback signal f equals the setpoint signal r and the deviation signal e of Hypothalamus 7 becomes zero. The body temperature tc reaches a new equilibrium state, say 38.4° C. Heat Generation System 4 responds to the viral disturbance action d by increasing the body temperature tc and counteracting to the virus with the reaction m of Heat Generation System 4 to protect its host from infection, suppress viral growth, and create an environment that helps inactivate viruses or impair the ability of the virus to penetrate and replicate inside living cells.

The severity of a viral infection depends on many factors related to the type, strains of viruses, viral load, number of viruses. Some viruses replicate extremely fast, and a higher viral load means more infected cells. Therefore, a more severe infection in the upper respiratory and GI tracts caused by Viral Infection 8 causes an even greater disturbance effect by action d, disrupting the vital activity of a greater number of cells and the functioning of certain mechanisms of Heat Generation System 4. The disturbance information signals td and tds increase even more. The feedback signal f of Thermoreceptor Sensory Cells 5 decreases. In turn, the deviation of the signal e increases. The output signal g of Thermoregulators 3 changes gradually and continuously, but more quickly due to the greater difference between the signals r and f, which can be associated either with a more dangerous virus or with a larger number of viruses. The human body temperature tc increases faster and becomes higher until the feedback signal f equals the setpoint signal r of Hypothalamus 7 and the deviation signal e becomes zero. The body temperature tc will settle at a new equilibrium state, for example at 39.6° C. The immune system of Heat Generation System 4 counteracts the viral infection action d of Viral Infection 8 by the reaction m that includes rising body temperature tc to protect the host from infection, suppress viral growth, and help kill viruses or weaken the ability of the virus to penetrate and replicate inside living cells.

### DETAILED DESCRIPTION OF THE INVENTION

The method of treating and preventing viral infections caused by cold, flu, acute respiratory viral infections, COVID-19 viruses, etc. is based on the fact that the survival time of the virus depends on temperature and on the results of scientific research that the virus can be inactivated/killed and/or its ability to penetrate and replicate inside living cells can be weakened under conditions of elevated temperature, which should be higher than the normal human body temperature.

Creating such an environment in the upper respiratory and GI tracts and preventing the spread of the virus to the lower respiratory tract through constant long-term consumption of hot water, prevents the development of pneumonia and accelerates recovery.

The agent of delivering heat is hot, drinking, pre-boiled water—the simplest, least expensive and most natural product for humans. This method additionally reduces the risk of pneumonia by washing viruses from the respiratory tract into the GI tract. At the same time, hot water prevents the development of viral infections in the GI tract as well as side complications. Drinking hot water helps the metabolism and immune processes fight infections more efficiently, which in turn can: (1) increase metabolic rate similar to how fever works that increases metabolic rate by 10-15% per degree Celsius, (2) improve blood circulation, ensuring oxygen and nutrients reach immune cells faster, and (3) help loosen mucus, making it easier to expel viruses from the respiratory tract, soothe a sore throat, reduce nasal congestion, improve coughing effectiveness to remove infected mucus, efficient nutrient transport for immune cells, faster removal of viral waste products through urine and sweat, better digestion, which supports metabolic energy production, and prevent dehydration, which is common during fever.

The present method prevents or reduces the spread of the virus into the lower respiratory tract and prevents the development of pneumonia most effectively in the early stages of the disease, when the infection is just beginning to develop in the upper respiratory and GI tracts, when the first signs of the viral infection appear.

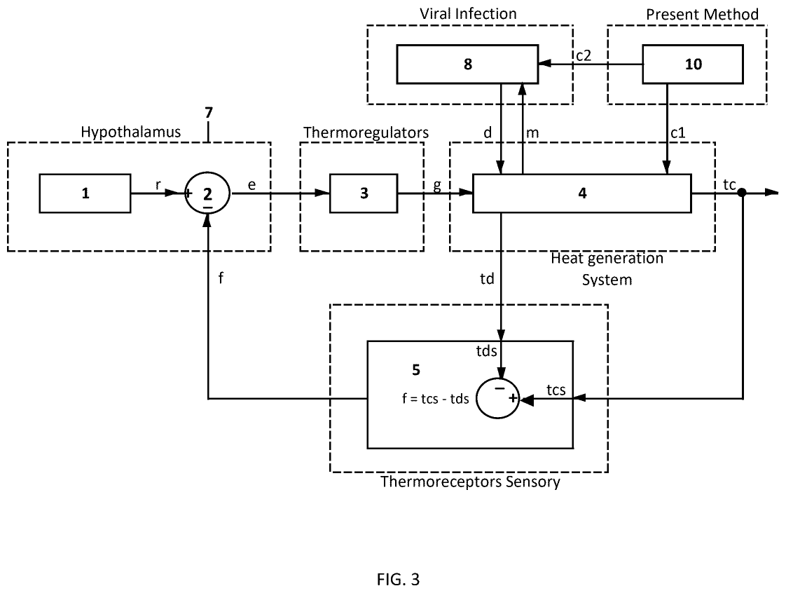

### DESCRIPTION OF THE FIG. 3

FIG. 3 shows the feedback control system of FIG. 2 with the disturbance compensator 10 provided by the present inventive method added in the block-diagram. As such, FIG. 3 shows a feedback control system with a conventional I controller, an external disturbance source (the viral infection), and the present method as a disturbance compensator.

### Model of Body Temperature Regulation in Viral Disorder and the Use of Present Method

FIG. 3 shows a simplified model of human body temperature regulation system under the influence of a viral infection as a foreign pathogen with the disturbance compensator block 10, which is the present method of treating viral infection. Present method 10 as the disturbance compensator delivers thermal energy c1 to Heat Generation System 4, and c2 directly to the external disturbance source, Viral Infection 8.

The prolonged, continuous, uninterrupted use of hot drinking water, preferably pre-boiled, according to Present Method 10 counteracts viral infections for at least two reasons:

First, Present Method 10 delivers thermal energy (i.e., heat) to the upper respiratory and GI tracts (where there is a high concentration of viral infection) using hot water with a maximum temperature set to a personal maximum water temperature (PMWT). The PMWT will be described in other sections. The consumption of hot water by the patient (also referred to herein as the subject) increases the patient's temperature in the upper respiratory tract by administering thermal energy. This method, represented by the disturbance compensator of Present Method 10 in FIG. 3, affects Viral Infection 8 by adding thermal energy c2 and thereby reduces the effect of the Viral Infection action d on Heat Generation System 4.

Second, prolonged and continuous use of the Present Method 10 reduces the number and virulence of viruses by adding thermal energy c1 and thereby helps the heat production of the immune system m, when counteracting the viral infection. At the same time, the consumed hot water improves metabolism and blood circulation. The purpose of this method is to kill as many viruses as possible and/or weaken the virus' ability to penetrate and multiply inside living cells using thermal energy and to enhance the functioning of the immune system.

As a result, over time, prolonged thermal dynamics of heat transfer in the feedback thermoregulation system of FIG. 3, Present Method 10 increases the temperatures in the upper respiratory tract and gastrointestinal tract and boosts the heat production of the immune system. The impact d of Viral Infection 8, that disrupts the normal functioning of the human body temperature regulation system, is reduced. The temperature disturbance signal td and the information signal tds decrease, thereby accelerating the process of returning the temperature to normal values, since the feedback signal f increases, due to the opposite value of the information signal tds. The comparator 2 of Hypothalamus 7 triggers the deviation signal e in the opposite direction and the deviation signal e decreases in accordance with the increase of the negative feedback signal f. The output signal g of Thermoregulators 3 decreases gradually and continuously with a rate of change determined by the integration time constants and depends only on the difference between the signals r and f. This signal g enters Heat Generation System 4, activates heat-loss mechanisms, the external signs of which may be sweating, skin cooling, frequent urination (release of warm liquid) and reduces the human body temperature tc, thereby restoring the body temperature tc in accordance with the homeostatic setpoint temperature, the normal functioning of the body.

The disturbance compensator of the Present Method 10 during long-term consumption of hot water helps the immune system to counteract the influence of Viral Infection 8 and by thermal energy c2 reduces the action d of Viral Infection 8 on Heat Generation System 4 by killing viruses and/or weakening their ability to penetrate and replicate copies inside the living cells. However, over time, after prolonged consumption of hot water, thermal energy c1 reduces (unloads) the heat production of the immune system's response m in the fight against Viral Infection 8. The immune system consumes a lot of energy to fight viruses. Present Method 10 compensates for part of the energy consumption of a weakened organism, necessary for the functioning of the immune system.

The temperature tc decreases until the feedback signal f equals the setpoint signal r of Hypothalamus 7 and the deviation signal e equals zero. The body temperature tc will reach a new equilibrium state, for example, it will return to a temperature of 37.2° C. from 39.6° C.

The longer the hot water is consumed and the higher the personal maximum water temperature (PMWT), the more viruses can be inactivated, the greater the likelihood that the body temperature tc will return to normal, and the human body temperature regulation system will return to the conventional block diagram described in FIG. 1. The body temperature tc is a good indicator of the severity of the disease and the correctness of the application of this method.

In order for the treatment of viral infections to be effective, detailed instructions for using this method are provided below.

### Instructions for the Use of this Method

As mentioned above, in the proposed method, each patient or subject should drink hot water in accordance with their unique physical constitution, one of the main parameters of which is the subject's body weight. This method is intended for the treatment of healthy adults infected with a viral infection with common body weights of about 50 kg to about 100 kg. However, it is appreciated that the method also can be used for a wider range of body weights. For people with underlying health conditions, the calculations may be determined by a physician and can be changed depending on the patient's health condition, especially for older patients with kidney problems to prevent hyponatremia.

According to the present method for substantially continuously, uninterrupted treatment, the hot water consumption rate (HWCR) may be determined using the formula:

$$HWCR = 0.34 mL / min / kg \times UW kg + 14 mL / min \pm 1 mL / min \qquad ( 3 )$$

$$where :$$

$$0.34 mL / min / kg is the hot water consumption rate per one kilogram of user weight ;$$

$$UW is the user ’ s weight in kilograms ;$$

$$14 mL / min is constant consumption rate compensation for minimal user weight ;$$

$$and$$

$$\pm 1 mL / min is a tolerance that defines the permissable deviation in the consumption rate .$$

For example, for a subject with a body weight of 50 kg, HWCR=31 ml/min±1 ml/min. In another example, for a subject with a body weight of 100 kg, HWCR=48 ml/min±1 ml/min, and for a subject with a body weight of 70 kg, HWCR≈38 ml/min±1 ml/min.

In the Journal of Food Science articles “Preferred Drinking Temperatures for Coffee and Tea” which discusses how a person can consume hot beverages while avoiding the possible risk of scalding depending on the temperature, and in the practical observations on human heat tolerance, it is understood that a typical person can drink water at a temperature of 45-50° C., which feels hot in a normal mouthful but is generally tolerable. Furthermore, it is understood that a typical person can drink water at a temperature of 55-60° C., which may feel very hot, but the person can drink the water in smaller sips that are less than a normal mouthful. Water temperatures above 65° C., which can potentially cause scalding, can be consumed in very small sips. Accordingly, it can be seen that the smaller the sip, the higher the temperature at which beverages can be consumed. However, according to the present method, the sip cannot be too small nor the temperature too high, as this may reduce the efficiency of the heat transfer while increasing the risk of scalding.

The present invention presents a method for determining the volume of a sip of hot water consumption, which, on the one hand, ensures an effective thermal effect on the body of a specific patient, and on the other hand, ensures safe conditions for consuming hot water. In this method, the volume of hot water in a small sip (VWSS) is determined by the formula:

$$VWSS = 0.06 mL / kg \times UW kg + 2 mL \qquad ( 4 )$$

$$where :$$

$$0.06 mL / kg is the volume of water of the small sip per one kilogram of user ’ s weight ;$$

$$UW is the user ’ s weight in kilograms ;$$

$$and$$

$$2 mL is constant sip volume compensation for minimal user weight .$$

For example, for a patient with a body weight of 50 kg, VWSS=0.06 mL/kg×50 kg+2 mL=5 mL, for a patient with a body weight of 100 kg, VWSS=8 mL, and for patient with a body weight of 70 kg, VWSS=6.2 mL.

The selected volume range within 5-8 mL per sip can be measured by using a means for measuring small volumes of water, such as a syringe, measuring cylinder or standard teaspoon, the capacity of which is usually taken to be equal to 5 milliliters (ml). The sip determination procedure is performed once before the treatment procedure. The calculated volume of water of the small sip for each patient helps prevent scalding and limits a significant increase in the rate of the hot water consumption, maintaining it in the desired range.

The above defined HWCR and VWSS allow users to define the time interval between sips (IBS) accordingly. In this method, the duration of time for one cycle of small sips (the period of time between the starts of two consecutive sips) minus the time of the sip itself is the inter-sip interval and is determined by the formula:

$$IBS = VWSS / HWCR - 2 seconds where swallowing a small sip of hot water at the personal maximum temperature takes approximately 2 seconds . \qquad ( 5 )$$

For example, for a patient with a body weight of 50 kg, IBS=5 mL×60 sec/31 mL−2 sec=9.7 sec−2 sec=7.7 sec; for a patient with a body weight of 100 kg, IBS=8 mL×60 sec/48 mL−2 sec=10 sec−2 sec=8 sec; and for patient with a body weight of 70 kg, IBS=6.2 mL×60 sec/38 mL−2 sec≈9.8 sec−2 sec=7.8 sec. These intervals between sips can be changed in case of uneven consumption of the hot water, for example, in case of loss of time in preparing the next portion of the hot water during long-term treatment with this method.

In some embodiments, the minimum temperature of hot water is 55° C., the use of which in small sips as specified above guarantees safe consumption conditions. But to ensure effective thermal impact by this method, the water temperature is defined as the personal maximum water temperature (PMWT), the highest temperature of hot water that the patient can drink in small sips without scalding. The present invention presents the safest procedure for determining the personal maximum water temperature (PMWT), in which the temperature determination procedure begins with drinking hot water with a minimum temperature, which in this method is 55° C.

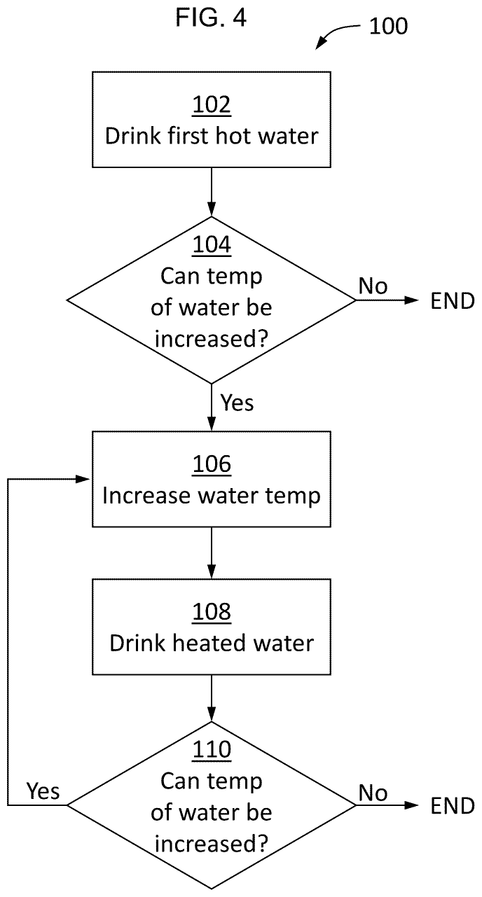

In some embodiments, as shown in FIG. 4, the procedure of determining the personal maximum water temperature (PMWT) includes the following steps 100.

At 102, the patient drinks at least 2 sips of the hot water at the specified minimum water temperature of 55° C. and with the sips at the determined small sip volume.

At 104, the patient determines if he/she may take sips larger than the specified small sip volume with the water temperature at 55° C. without discomfort (e.g., without feeling scalded). If yes, then the patient moves to step 106. If no, the determination ends, and the patient's Personal Maximum Water Temperature (PMWT) is set to 55° C.

At 106, because the patient has not yet reached his/her PMWT, the temperature of the water may be increased (e.g., by adding water of a higher temperature to the water), and at 108 the patient may drink at least 2 sips of the water at the increased temperature with the sips at the determined small sip volume.

At 110, the patient determines if he/she may take sips larger than the specified small sip volume with the water at the increased temperature without discomfort (e.g., without feeling scalded). If yes, then the patient returns to step 106. If no, the determination ends, and the patient's Personal Maximum Water Temperature (PMWT) is set to the increased temperature.

In general, the process described above results in determining the patient's PMWT wherein the patient can only drink the specified small sip volumes without scalding (that is, if larger sips at the increased temperature were taken then the patient would feel scalded).

This temperature of the water may be measured using a standard instant-read beverage thermometer or similar. The personal maximum water temperature (PMWT) is determined by the patient once at the beginning of the method, and it is preferable to use means for maintaining the temperature of the hot water at this temperature, such as double-walled mugs, for effective heat exposure.

The minimum duration of hot water consumption (MNDC), according to the present method, is determined using the formula below.

$$MNDC = 5850 min \times ° C . / PMWT ° C . \qquad ( 6 )$$

$$where :$$

$$5850 is constant coefficient in ( minutes \times degrees Celsius ) ;$$

$$and$$

$$PMWT is a personal maximum water temperature in degree Celsius .$$

For example, for PMWT=55° C., MNDC=106 minutes; for PMWT=75° C., MNDC=78 minutes; and for PMWT=65° C., MNDC=90 minutes.

The maximum duration of hot water consumption (MXDC) is defined as:

$$MXDC = ( MNDC + 45 min ) minutes \qquad ( 7 )$$

$$where :$$

$$MNDC is minimum duration of hot water consumption in minutes ;$$

$$and$$

$$45 is a constant number in minutes .$$

For example, for PMWT=55° C., MXDC=151 minutes; for PMWT=75° C., MXDC=123 minutes; and for PMWT=65° C., MXDC=135 minutes.

The above defined MNDC and HWCR allow users to calculate the minimum volume of hot water consumption (MNVC) using the formula:

$$MNVC = MNDC \times HWCR \qquad ( 8 )$$

For example, for MNDC=106 minutes and HWCR=31 mL/min, MNVC=106 min×31 mL/min=3286 ml; for MNDC=78 minutes and HWCR=48 mL/min, MNVC=78 min×48 mL/min=3744 mL; and for MNDC=90 minutes and HWCR=38 mL/min, the person should drink minimum volume of hot water MNVC=90 min×38 mL/min=3420 mL.

The above defined MXDC and HWCR allow users to calculate the maximum volume of hot water consumption (MXVC) using the formula

$$MXVC = MXDC \times HWCR \qquad ( 9 )$$

For example, for MXDC=151 minutes and HWCR=31 mL/min, MXVC=151 min×31 mL/min=4681 ml; for MXDC=123 minutes and HWCR=48 mL/min, MXVC=123 min×48 mL/min=5904 mL; and for MXDC=135 minutes and HWCR=38 mL/min, the person can drink maximum volume of hot water: MXVC=135 min×38 mL/min=5130 mL.

The instruction below illustrates the use of this method for a patient weighing 70 kg and with a determined personal maximum water temperature (PMWT) of 65° C.

In this example, for the MNDC=90 minutes of treatment with HWCR=38 mL/min, the patient should drink a minimum volume of hot water of 38 mL/min×90 min=3420 mL. For the MXDC=135 minutes and the same HWCR=38 mL/min, the person should drink 38 mL/min×135 min=5130 mL. The entire process of uninterrupted consumption of hot water is carried out in small sips of approximately 6 mL.

For the treatment of a viral infection, the temperature, the duration of time and the volume of continuous, uninterrupted consumption of hot pre-boiled drinking water are critical parameters for heat therapy of each patient with this method.

Next, after the parameters described above are determined for the particular patient, the patient may start treatment using the present method. During 90 minutes of continuous consumption, at a consumption rate of 38 mL/min, and without interruptions, the patient should drink 3420 mL of hot water, but not less, to treat the viral infection.

In some embodiments, the process of treating viral infections should last at least until the minimum duration of hot water consumption (MNDC) is reached, and may continue with the same rate of water consumption, wherein the end of the treatment process is determined by objective measures, including a decrease in body temperature and stabilization below 37.3° C., a reduction in sore throat below 2 points on a scale of 0 to 10, and/or a reduction in muscle aches below 2 points on a scale of 0 to 10, and/or a reduction headache below 2 points on a scale of 0 to 10, and/or normalization of individual's pulse rate; and/or subjective indicators that indicate to the patient either a complete disappearance of symptoms or a significant reduction in symptoms, e.g., no longer experiencing chills, relief of nasal or chest congestion, relief of cough, ease of removing infected mucus, more comfortable breathing, feeling less tired and more energetic, and improvement in general well-being of at least 70%.

If the patient, according to the objective measures and subjective indicators mentioned above, no longer experiences the initial symptoms after 90 minutes of drinking hot water, then there may be no need to drink more than 3420 mL of water. However, if the patient continues to experience the same symptoms, the patient can continue the treatment and drink more than 3420 mL of hot water.

Symptoms are indicators of illness. Without symptoms, a person may not know they are sick. Even with symptoms, a person may not know if the symptoms are caused by a cold, a flu, or coronaviruses. Each patient experiences and evaluates symptoms uniquely and subjectively, even common symptoms.

However, in the present method, subjectivity, regarding symptoms, is limited by the requirement to consume a minimum of 3420 mL and a maximum of 5130 mL of hot water for a patient weighing 70 kg. Within the range of 90 minutes to 135 minutes and volumes from 3420 mL to 5130 mL, the final duration and volume of hot water consumption are determined by the disappearance or significant reduction of symptoms according to objective measurements and subjective indicators.

If, after drinking 3420 mL of hot water, the patient still feels the same symptoms as before the treatment, the patient can continue the treatment with this method until he/she feels that the symptoms have disappeared or have significantly decreased according to objective measurements and according to the patient's subjective assessment. In any case, water consumption should not exceed a maximum duration of 135 minutes and/or a maximum volume of 5130 ml.

Symptoms may be used as an indicator of the end of the treatment process by this method. The uniqueness of the application of this method is that the temperature, time and volume of consumption are selected and controlled by the patient himself for healthy adults or by a doctor when the patient's health condition requires such medical consultation. In most cases, the patient him/her-self determines the volume within the range from 3420 mL to 5130 mL of hot water consumption, the duration of treatment within the range from 90 minutes to 135 minutes, and when the process may end.

Sometimes the relief of symptoms occurs earlier than the inactivation of viruses in the upper respiratory and GI tracts, but sometimes later. For example, fever may begin to subside after 1 hour of treatment, while muscle pain may go away after 2 hours of treatment. In any case, a person weighing 70 kg and with a maximum water temperature of 65° C. should drink hot water for at least 90 minutes and at least 3420 mL of the hot water, while not exceeding a maximum of 135 minutes and 5130 mL, respectively.

### Use of the Invention

### Treatment of a Viral Infection

This method is most effective for treatment in the early stages of infection, when the first symptoms appear. If a person weighing 70 kg develops symptoms such as fever, sore throat, shortness of breath, muscle pain, etc., the patient should immediately begin treatment using this method. The sooner the patient begins treatment, the higher the chance of a complete cure.

### Treatment Procedure

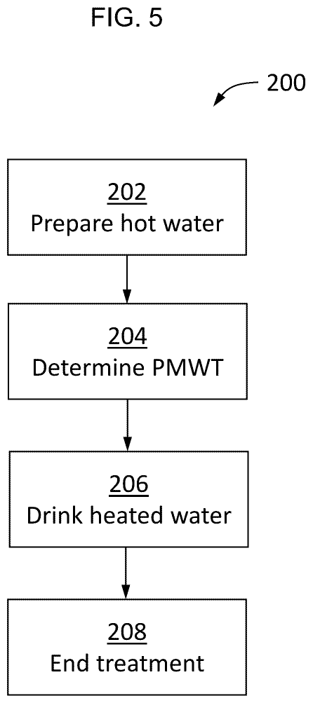

In some embodiments, as shown in FIG. 5, Treatment Procedure of a viral infection using the present method includes the following steps 200.

As a first step at 202, the patient may boil ordinary drinking water, for example in a kettle, pour the heated water into a cup/mug, preferably double-walled, and let it cool and/or dilute the hot water with cooler water until the hot water reaches a temperature at which it can be safely consumed, e.g., based on the person's ability to drink hot water, but not below 55° C.

As a second step at 204, the patient may then try drinking the hot water at the chosen temperature. If the patient finds that the sip volume used can be significantly larger than the volume of a standard teaspoon (5 mL), then the water temperature is too low, and the patient may increase the temperature of the water, e.g., by adding hotter water to the water. The patient may try sipping the newly heated water again, and may repeat the process until the temperature of the water in the cup/mug allows the patient's sip volume to be slightly more than 1 regular teaspoon without scalding, as the sip volume should only be approximately 6 mL. This temperature may then be set as the patient's personal maximum water temperature (PMWT) that a patient can drink. The PMWT is preferably determined only once. For demonstrational purposes, the PMWT may be 65° C.

As a third step at 206, the patient may then start the process of drinking the hot water at the PMWT. If circumstances do not allow determining the personal maximum water temperature using the above-mentioned process (for example, a patient is at work or on vacation far from home), then the patient can simply drink pre-boiled, hot water with the highest temperature that he/she can consume in small sips without scalding; or use your normal method of drinking hot drinks. Pour boiling water into a cup and let it cool slightly. As usual, try to take a couple of very small sips. If the temperature is too high to drink, wait until it cools down or dilute with colder boiled water. Then try to drink in small sips again. If the temperature allows you to drink the water without scalding, in sips of a given volume, then this is your maximum water temperature. If the water temperature still does not allow you to drink in a given sips, then continue to cool the water until the temperature allows you to drink in a given sips without scalding.

It is advisable to drink water at a constant, even pace with an interval between sips of approximately 8 seconds in accordance with the calculations given above.

After the first cup/mug has been drunk, the patient may continue to drink the hot water (at the personal maximum water temperature (PMWT) with minimal breaks between cups/mugs to ensure a continuous, even rate of consumption. If the drinking process is interrupted for any reason, the intervals between sips can be adjusted accordingly.

As a fourth step at 208, the patient may continue the process and furthermore complete the treatment process when (1) the duration of hot water consumption is at least 90 minutes, (2) the volume of hot water consumption is at least 3420 mL, and (3) the symptoms have disappeared or have significantly decreased in accordance with the above objective measurements and subjective indicators.

If the patient continues to experience the symptoms that were present before using this method, the patient may continue to consume hot water at the same rate, the duration of hot water consumption may be more than 90 minutes, and the volume of consumption may be more than 3420 mL until the symptoms disappear or are significantly reduced according to the above measurements and indicators.

If the duration of the hot water consumption reaches 135 minutes, and the volume of hot water consumed reaches 5130 mL, in any case, regardless of whether the symptoms have disappeared or reduced, the method may end.

The minimum duration and minimum volume of hot water consumption are mandatory requirements of this treatment method. The end of a longer treatment than the minimum duration and correspondingly larger volume of hot water consumption than the minimum volume is determined by the symptoms disappearing or significantly weakening, while the maximum duration and maximum volume of hot water consumption are also mandatory requirements of this treatment method.

### Prevention of a Viral Infection

The present method also may be used to prevent viral infections of the upper respiratory tract. The method for this purpose also includes continuous, uninterrupted exposure of heat to the upper respiratory and gastrointestinal tracts by consuming the pre-boiled, hot water with a specified Personal Maximum Water Temperature (PMWT), while the minimum duration of hot water consumption is 30 minutes, and the maximum duration of hot water consumption is 60 minutes. The exact duration of hot water consumption between the minimum and maximum durations may be selected individually by each patient, depending on all the circumstances that determined the use of this method of preventing viral infections.

For example, the method may be used for prevention when a person believes that he or she has been in a contaminated environment (for example, near sick people). The prevention method should be used in the same way as other proactive measures determined by health professionals, such as hand washing, using hand sanitizer, or wearing a face mask. This method may be used more frequently during virus seasons.

### Prevention Procedure:

The patient may repeat steps 1 to 4 (202-208) of the above Treatment Procedure, but with a range of hot water consumption duration from 30 minutes to 60 minutes and a range of hot water volume consumed from 1140 mL to 2280 mL (e.g., for a person weighing 70 kg and PMWT=65° C.). The exact duration and volume of hot water consumption within the specified limits is determined by the person him/her-self, depending on the reason for use.

### Application

Depending on the general health condition, physiological characteristics of the particular patient, and the external environment, treatment and prevention procedures can be used in different combinations, with different frequencies, during the day, days, weeks or months. The effectiveness of treatment with this method depends on the time of its use. The later from the onset of symptoms the infected person begins treatment, the less effective this method is. It is most effective in the early stages of the disease and can speed up recovery and prevent complications. This method may not treat bronchitis or pneumonia.

The present invention can be used as an independent method for the treatment and prevention of viral infections, as well as an addition to other existing traditional methods.

This method is safe and has no side effects. However, people with special medical conditions should use this method with the permission of their doctor or under doctor supervision. Each patient is personally responsible for using this method properly and in accordance with his/her physiological characteristics and medical condition.

Time Frame for Using this Method

### Example 1

### First Day:

11:00 AM. A person takes their temperature for the first time because they feel unwell. The temperature is 39.5° C. (103.1° F.).

11.15 AM. The person begins to drink hot boiled water according to the Treatment Procedure described above.

6:00 PM. The person measures their temperature again. If the temperature is significantly below 39.5° C. (103.1° F.), for example 37.2° C. (99° F.), then no further action is required using this method that day. If the temperature is above 38° C. (100.4° F.), the person may use the Prevention Procedure described above after 6:00 PM that day.

### Second Day:

If by 6 PM the temperature has not increased, treatment with this method is complete. If during the day the temperature has risen significantly, the Treatment Procedure can be repeated again.

After using this method for two days, further use of this method can be terminated. In addition, under all circumstances, the generally accepted treatment methods should be followed properly, including seeking medical help as necessary.

### Example 2

### First Day:

5:00 PM. A person takes their temperature for the first time because they feel unwell. The temperature is 40.5° C. (105° F.). In this case, the person may take fever reduced medicine and, at 5.30 PM, the person may begin to drink hot boiled water according to the Treatment Procedure described above.

10.00 PM. The person measures their temperature again. If their temperature is significantly below 40.5° C. (105° F.), such as 37.2° C. (99° F.), no further action is required regarding the method that day. If the temperature is above 38° C. (100.4° F.), the person can take a fever reducer or multi-symptoms relief medicine and go to bed.

### Second Day:

If by 6 PM the temperature has not increased, treatment with this method is complete. If during the day the temperature has risen significantly, the Treatment Procedure can be repeated again.

After using this method for two days, if the patient's temperature is slightly higher than normal temperature, the Prevention Procedure can be repeated. If the temperature is still high, conventional treatment methods, including medical care, should be followed accordingly.

### Test Results

### Example 1

This method was first tested for the treatment of flu. When mild symptoms of sore throat and muscle pain appeared, the patient immediately began to follow the treatment procedure of this method as described above. The patient achieved complete recovery without any initial symptoms within one day.

### Example 2

This method also was tested for the treatment of suspected coronavirus COVID-19. When the symptom of high fever of 40.5° C. appeared, the person immediately started to follow the treatment procedure of this method as described above. The person's temperature returned to normal within one day. Later, a cough appeared. The patient was isolated for the next 5 days to avoid infecting other people, since the patient continued to have a mild cough. After two weeks of coughing, the patient was given a chest X-ray to make sure that the infection did not develop into pneumonia, which it had not.

The present method also was used to treat a variety of other people with symptoms including fever, muscle pain, headache, weakness, runny nose, sore throat. In each case, most symptoms disappeared or were significantly reduced after one or two days of treatment. All the above treatment parameters and their limits of use were empirically obtained during 10 years of experimentation by the inventor, who tested patients with colds, flu and, presumably, Covid-19. Every person who used this method recovered quickly and without any complications.

The significant advantages of the proposed method of treatment and prevention of viral infections caused by viruses whose survival time depends on temperature are multifold. Correct and timely application of the treatment and preventive procedures shows the high efficiency of the proposed method. This method is easily accessible, inexpensive, and safe, thereby providing a simple and easy to follow method of treatment and prevention of viral infections. This method can help prevent epidemics or pandemics by slowing the spread of viral infections such as colds, flu, acute respiratory viral infections, COVID-19, etc. Recognition of this method by the scientific community can make this method one of the most popular methods of treatment and prevention of viral infections worldwide. Meanwhile, the study of the effect of temperature, including hot water on viruses should be continued, expanded and carried out in full, including comprehensive clinical and statistical studies. Today, the creation of vaccines or antiviral drugs is the only way to combat viruses. A better understanding of the effect of temperature on the virus inside the human body, including prolonged continuous thermal exposure of the virus in the upper respiratory and GI tracts, through consumption of hot drinking pre-boiled water, and the application of this method are also needed to guide future efforts in the treatment and prevention of viral infections.

Where a process is described herein, those of ordinary skill in the art will appreciate that the process may operate without any user intervention. In another embodiment, the process includes some human intervention (e.g., a step is performed by or with the assistance of a human).

As used in this description, the term “portion” means some or all. So, for example, “A portion of X” may include some of “X” or all of “X”. In the context of a conversation, the term “portion” means some or all of the conversation.

As used herein, including in the claims, the phrase “at least some” means “one or more,” and includes the case of only one. Thus, e.g., the phrase “at least some ABCs” means “one or more ABCs” and includes the case of only one ABC.

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

As used herein, including in the claims, a list may include only one item, and, unless otherwise stated, a list of multiple items need not be ordered in any particular manner. A list may include duplicate items. For example, as used herein, the phrase “a list of XYZs” may include one or more “XYZs”.

It should be appreciated that the words “first” and “second” in the description and claims are used to distinguish or identify, and not to show a serial or numerical limitation. Similarly, the use of letter or numerical labels (such as “(a)”, “(b)”, and the like) are used to help distinguish and/or identify, and not to show any serial or numerical limitation or ordering.

No ordering is implied by any of the labeled boxes in any of the flow diagrams unless specifically shown and stated. When disconnected boxes are shown in a diagram, the activities associated with those boxes may be performed in any order, including fully or partially in parallel.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of treating a viral infection in a human subject in need thereof, the viral infection caused at least by a contagious respiratory coronavirus related to severe acute respiratory syndrome (SARS) coronavirus whose survival time is temperature dependent, the method comprising:
administering to the subject an amount of thermal energy from a liquid product using a predetermined combination of:
(i) a temperature of the liquid product,
(ii) a duration of time of the administrating the thermal energy from the liquid product, and
(iii) an amount of the liquid product,
wherein:
the temperature of the liquid product is at least 55° C.; and
the liquid product is at least pre-boiled drinking water.

2. The method of claim 1, wherein the thermal energy is administered orally to an upper respiratory tract and a gastrointestinal tract of the subject by having the subject drink the amount of the liquid product.

3. The method of claim 2, wherein the subject drinks the amount of the liquid product at a consumption rate, wherein the consumption rate is based on a body weight of the subject.

4. The method of claim 3, wherein the consumption rate is determined by adding a first constant value to a product of the body weight of the subject and a second constant value, wherein the first constant value is 14 mL/min, and the second constant value is 0.34 mL/min/kg.

5. The method of claim 4, wherein the subject drinks the amount of the liquid product in sips, each sip having a sip volume, wherein the sip volume depends on the body weight of the subject and is determined by adding a third constant value to a product of the body weight of the subject and a fourth constant value, wherein the third constant value is 2 mL, and the fourth constant value is 0.06 mL/kg.

6. The method of claim 5, wherein the subject drinks the sips sequentially with an interval between each sequential sip, wherein the interval is determined by subtracting a fifth constant value from a quotient of the sip volume and the consumption rate, wherein the fifth constant value is 2 seconds.

7. The method of claim 6, wherein the temperature of the liquid product is determined as a maximum temperature of the liquid product having the highest temperature which the subject drinks of the liquid product in sips having the predetermined sip volume without causing scalding of the subject, the maximum temperature is greater than or equal to a sixth constant value, wherein the sixth constant value is 55° C.

8. The method of claim 7, wherein the subject drinks the amount of the liquid product for at least a minimum duration of time based on the maximum temperature of the liquid product.

9. The method of claim 8, wherein the minimum duration time is determined by dividing a seventh constant value by the maximum temperature of the liquid product, wherein the seventh constant value is 5850 min x° C.

10. The method of claim 9, wherein the subject drinks the amount of the liquid product for no longer than a maximum duration of time determined by adding an eighth constant value to the minimum duration of time, wherein the eighth constant value is 45 min.

11. The method of claim 10, wherein the amount of thermal energy administered to the subject is based on the product of the maximum temperature of the liquid product and the amount of the liquid product, wherein the amount of the liquid product is a volume of the liquid product.

12. The method of claim 11, wherein the subject drinks the liquid product for at least the minimum duration of time, during which the subject drinks at least a minimum volume of the liquid product, wherein the minimum volume of the liquid product is determined by multiplying the minimum duration of time by the consumption rate.

13. The method of claim 12, wherein the subject drinks the liquid product for no longer than the maximum duration of time, during which the subject drinks no more than a maximum volume of the liquid product, wherein the maximum volume of the liquid product is determined by multiplying the maximum duration of time by the consumption rate.
